(19) United States
(12) Patent Application Publication
(10) Pub. No.: US 2022/0160225 A1
(43) Pub. Date: May 26, 2022

(54) Multi-Optotype, Computerized Visual Acuity Examination with Voice Assistance

(71) Applicant: Soham Vinayak Govande, Round Rock, TX (US)

(72) Inventor: Soham Vinayak Govande, Round Rock, TX (US)

(73) Assignee: Soham Vinayak Govande, Round Rock, TX (US)

(21) Appl. No.: 17/105,610

(22) Filed: Nov. 26, 2020

(51) Int. Cl. A61B 3/032; G06F 3/0482; H04R 1/02; G06F 3/16; A61B 3/00

(52) U.S. Cl. CPC A61B 3/032; G06F 3/0482; H04R 1/028; H04R 2499/15; A61B 3/005; A61B 3/0033; G06F 3/167

(57) ABSTRACT

Systems and methods for assessing the visual acuity of a person using a consumer computing device are described. In this approach, the user is assisted by a helper (examiner). The method involves providing instructions, offering voice assistance, displaying optotypes for the user to identify, presenting an interface for the examiner to select the number of incorrect optotypes, determining acuity score(s), and presenting an interface comprising of the score(s) and their interpretation(s). The present invention improves upon current market solutions: it functions without network access (useful in infrastructure-limited regions), provides voice assistance in multiple languages, ensures precise optotypes by using a custom database of consumer computing device screen sizes, provides automatic calibration to a wide range of devices, and offers a selection of optotypes not found in other apps, such as those for non-literate and non-English speaking test-takers. Thus, this invention allows for the identification visual impairments at their early onset.

### CROSS-REFERENCE TO RELATED APPLICATIONS

N/A

### BACKGROUND OF THE INVENTION

### 1. Field of the Invention

The present invention is in the field of eye examination and pertains particularly to using a computerized method for visual acuity examinations (visual acuity exams).

### 2. Description of Related Art

In the field of optometry, visual acuity exams may be used to determine how well a person can see letters or symbols from a distance and discern the details of what they can see. Thus, visual acuity exams are necessary for people to know if they have a refractive visual impairment, such as myopia.

Traditionally, patients visit a nearby professional optometrist or ophthalmologist to measure their visual acuity. However, such eye care is often inaccessible to people in underserved regions due to factors including high costs, lack of transportation, and lack of nearby professionals. Furthermore, in regions where a single eye clinic may serve a broad area, it may be difficult to get an in-person appointment due to the high demand. This may exacerbate the visual impairment by preventing diagnosis at early onset.

Computerized, remote visual acuity examinations already exist, and in part, some present solutions mitigate a few of the aforementioned problems. However, the present inventors have observed that current computerized examinations are suboptimal due to several reasons. Firstly, some current examinations require fluency in reading English visual acuity characters (“visual acuity characters” are the figures one reads while taking a visual acuity exam and are hereafter referred to as “optotypes”), so non-literate individuals and non-English speakers may be unable to take these visual acuity exams; this makes such solutions impractical for use in remote areas with populations diverse in age and language. Secondly, studies have found that many optotypes are imprecisely scaled to the target device screen size, making the results inaccurate; ideally, a computerized visual acuity exam should provide accurate results on a wide range of device screens. Thirdly, some present solutions rely on the user's ability to look at and read directions from a device screen (directions on how to take a visual acuity exam), but users with severe visual impairment may not be able to do this. Fourthly, some current solutions claim to provide a remote human technician (a remote human technician is someone who provides real-time human assistance during a visual acuity exam communicating remotely through the app), but this approach can be impractical and costly in areas without a network connection. Fifthly, other computerized visual acuity exams require a network connection for other aspects of the invention, which can make these inventions unable to function in infrastructure-limited regions without network access.

Therefore, what is clearly needed is a computerized visual acuity examination that solves the problems mentioned above.

### BRIEF SUMMARY OF THE INVENTION

The present inventors have observed that current computerized methods for remotely measuring visual acuity are suboptimal in several ways, namely, in that they may not be useful to people who live in developing and infrastructure-limited regions. The World Health Organization estimates that 90% of visual impairments around the world occur in low- and middle-income households in developing countries; thus, being able to address the needs of this population is important. However, current market solutions are not able to do this due to the aforementioned problems. For example, current market solutions do not offer the user a selection of optotypes and lack multilingual voice assistance, resulting in a ‘one-size-fits-all’ process that does not sufficiently address the needs of diverse populations in developing countries. Also, current market solutions rely on a network to transfer data between the consumer computing device and a web server, but networks can be inaccessible in infrastructure-limited regions. Similarly, while current market solutions rely on a remote human technician to monitor the system and provide real-time assistance during eye examinations, in practicality, there may not be technicians available to help infrastructure-limited these areas, and the lack of network access may be a barrier to such communication. Finally, many present solutions do not accurately scale optotypes on a wide range of device screens and resolutions (some solutions may require the user to manually calibrate their device for this, while others simply use inaccurate optotypes).

The present disclosure may provide a solution to these problems by providing remote, computerized visual acuity examinations in a manner that addresses these problems. Remote visual acuity testing removes the need for the user to travel to a professional optometry clinic, and computerized visual acuity exams allow the user to use a consumer computing device (e.g., a smartphone, a tablet computer, or a laptop computer) to assess their visual acuity from a convenient location. Currently, computerized visual acuity examinations do exist, but current market solutions are inadequate as a public health measure; the present invention solves these problems in several ways. Firstly, the present invention provides a visual acuity examination without the need for network access on the consumer computing device. Also, the present invention provides a selection of optotypes so that non-literate individuals and non-English speakers can take the visual acuity exam. Furthermore, the present invention removes the need for remote human technician assistance by providing automatically-generated voice guidance (“automatically-generated voice guidance” may hereafter be referred to as “voice assistance”) to the user, in a language of their choosing, without the need of a remote human technician; this may help users who are too visually impaired to read written instructions. Additionally, the present invention creates more precise optotypes by using a custom database of consumer computing device screen sizes to determine the physical size (“physical size” refers to a size in the real world, measured in units such as inches or centimeters, rather than a “digital size” which is measured in pixels) of the current consumer computing device screen, and hence, more accurately determine the pixel-to-inches ratio to use when scaling optotypes on a wide range of consumer computing devices; this auto-calibration is performed automatically, rather than manually. Finally, the present disclosure differs from other apps in that the optotype size is incremented/decremented sequentially according to the row size of industry-standard eye charts (e.g. size of row 1 to size of row 2 to size of row 3), whereas other apps may change the optotype size non-sequentially (e.g. size of row 1 to size of row 9 to size of row 5). Thus, the present invention allows an accurate visual acuity examination to be taken by nearly any capable person on nearly any consumer computing device, even in remote and underprivileged areas where eye care professionals are unavailable.

In one embodiment of the invention, a method for testing the visual acuity of a user with the help of an examiner using a consumer computing device is described. The consumer computing device includes a display screen, a speaker, a computer processor, and a memory (e.g., a tablet computer, a smartphone, a laptop computer, etc.). The user is a human subject, and the examiner is a human assistant who does not need to be a professional in conducting vision screening. The method comprises the steps (a) initiating a visual acuity test to allow an examiner to assess the visual acuity of a user using a computerized consumer device, the computerized consumer device comprising a display screen, a computer processor, a speaker, and a memory, the user being a human subject and the examiner being a human assistant who does not need to be a professional in conducting vision screening; (b) instructing the user to be located a certain distance from the consumer computing device at eye level and optionally providing automatically-generated voice assistance without the need of a remote human technician; (c) presenting a visual acuity exam without use of a refractor lens assembly, wherein the visual acuity exam comprises displaying various optotypes on the screen of the consumer computing device for the user to identify, such that the optotypes are rescaled to occupy a calculated physical size; (d) presenting an interface (through the consumer computing device) through which the examiner can select the number of optotypes that were identified incorrectly; (e) repeating steps (d)-(e) between 1 and 25 times, each time modifying the optotype size based on the examiner's previous input of the number of incorrect optotypes; (f) repeating steps (c)-(e) for each eye or set of eyes under examination, such as the right eye, the left eye, and both eyes, and optionally performing the visual acuity exam twice for each eye or set of eyes; (g) determining visual acuity score(s), from 20/20 to 20/200, for each eye or set of eyes under examination; and (h) presenting an interface (through the consumer computing device) comprising of the said score(s), an interpretation of said score(s), and options to locate nearby professional optometry clinics, to save the score(s) to the nonvolatile memory of the consumer computing device, and to share the score(s) with another person(s). In one embodiment of the method, the optotypes used for the visual acuity examination process can be selected through a menu comprising of Snellen, ETDRS, Japanese (katakana), Korean (Hangul), Greek, Tumbling E, LEA Symbols and Landolt C. Also in one embodiment of the method, the language used for voice assistance can be selected through a menu comprising of English, Spanish, German, Marathi, and Hindi. Also in one embodiment of the method, the optotypes used for the visual acuity examination process are rescaled in size according to the consumer computing device's screen size, such that if the user has provided override values for the device physical width and height, those values will be used, and if the user has not provided override values, then the consumer computing device will search a database of device screen sizes (which is stored in the nonvolatile memory of the consumer computing device) using the consumer computing device's model number and manufacturer as a search query. Also in one embodiment of the method, the consumer computing device comprises a smartphone, tablet computer, or personal computer.

In another embodiment of the invention, a consumer-based system for carrying out a visual acuity test of a user is described, comprising a consumer device comprising a display screen, a computer processor, a speaker, and a memory. The computerized consumer device may comprise a display screen, a computer processor, and a speaker. The computer processor may be configured to execute steps (a)-(h) of the method summarized above.

In another embodiment of the invention, a non-transitory computer-readable medium comprising program instructions for permitting a consumer computing device to perform a visual acuity examination of a user with the help of an examiner is described. The computerized consumer device may comprise a display screen, a computer processor, and a speaker. The program instructions are configured to, when executed, cause a computer processor of the computerized consumer device to execute steps (a)-(h) of the method summarized above.

### BRIEF DESCRIPTION OF THE FIGURES

FIG. 1 illustrates an exemplary setup for conducting a two-person computerized eye examination with a consumer computing device according to an exemplary aspect, such that the device is being held by the examiner.

FIG. 2 illustrates another exemplary setup for conducting a two-person computerized eye examination with a consumer computing device according to an exemplary aspect, such that the device is placed on a surface.

FIG. 3 illustrates a flow diagram showing an exemplary approach of the commands that an application may accept and the instructions that it may follow using a consumer computing device according to an exemplary aspect

FIG. 4 illustrates an example main menu screen (a “screen” may also be referred to as a “page”) of a graphical interface (hereafter referred to as a “graphical user interface” or “GUI”) presented via a visual acuity examination application (or app) through which the examiner may input commands.

FIG. 5 illustrates an exemplary instructions screen of a GUI for a visual acuity exam presented via an app at a display screen of a computerized consumer device, such as a tablet or a smartphone, according to an example.

FIG. 6 illustrates an exemplary GUI screen of an app informing the user to cover a certain eye or uncover both eyes before conducting a visual acuity exam, according to an example.

FIG. 7 illustrates an exemplary GUI screen of an app for conducting a visual acuity exam, according to an example.

FIG. 8 illustrates a flow diagram showing an exemplary approach for carrying out a visual acuity exam through a visual acuity exam app using a consumer computing device, according to an exemplary aspect.

FIG. 9 illustrates an exemplary results GUI screen of an app displayed after the completion of visual acuity test(s), according to an example.

FIG. 10 illustrates an exemplary settings GUI screen of an app displayed upon request to modify the settings, according to an example.

FIG. 11 illustrates an exemplary optotype selection GUI screen of an app displayed upon request to select an optotype, according to an example.

FIG. 12 illustrates an exemplary language selection GUI screen of an app displayed upon request to select a language for voice assistance, according to an example.

FIG. 13 illustrates an exemplary GUI screen of an app displayed upon request to enter device screen size measurements, according to an example.

### DETAILED DESCRIPTION OF THE INVENTION

The present inventors have developed an approach for conducting visual acuity examinations (examinations that measure one's visual acuity) using a consumer computing device (i.e., a smartphone, personal computer, a notebook computer, a tablet computer, etc.). Visual acuity examination results are important to determine one's need for professional medical guidance. However, visual acuity examinations are often inaccessible in rural/low-infrastructure regions, and the need arises to be able to conduct such vision tests remotely on a large-scale, in contrast to an in-person eye examination. This may provide several advantages compared to traditional, in-person testing by providing real-time feedback on test results, offering accessibility to a much larger audience, and most importantly, a lower cost than in-person examinations. Furthermore, readily available visual acuity examinations may lead people to seek eye care more actively. Although certain previous inventions have created computerized visual acuity examinations, they are often suboptimal due to the reasons described in the Background section. Thus, the present inventors have created an invention to solve these problems. In the following figures, a dashed line comprising of equal-length dashes indicates a region bounded by a computerized consumer device screen, and a dashed line with a pattern of long- and short-length dashes indicates a region that a corresponding lead line is referring to (long- and short-length dashed lines may not be visible in the actual invention).

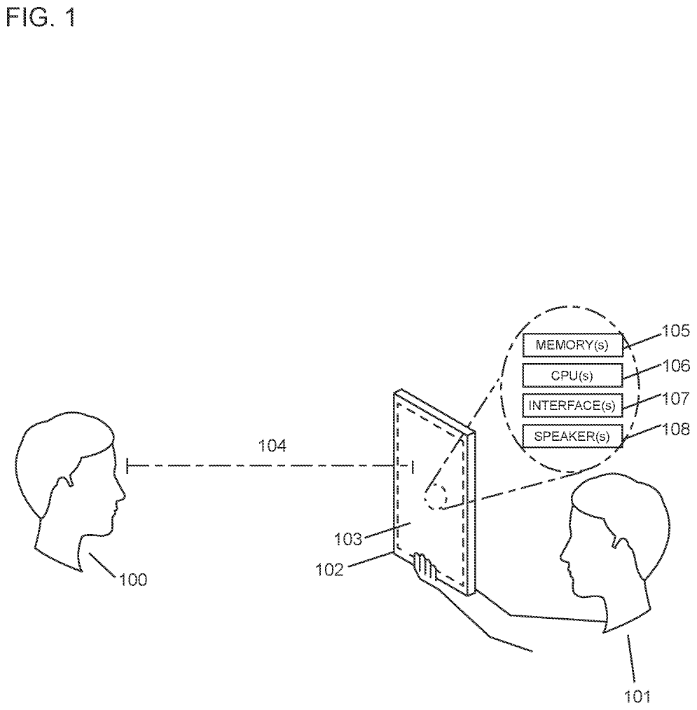

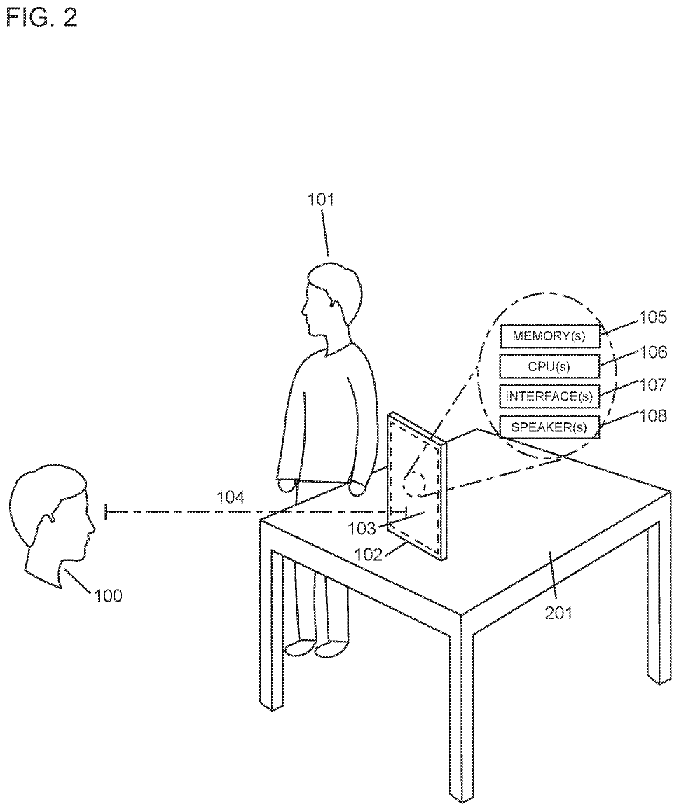

FIGS. 1-2 illustrate exemplary settings for conducting exemplary computerized visual acuity examinations. In one embodiment, FIG. 1 illustrates one such exemplary setting using a consumer computing device 102. As shown in FIG. 1, a consumer computing device 102 (i.e., a smartphone, personal computer, a notebook computer, a tablet computer, etc.) may have the functionality of conducting a visual acuity examination for a user 100, who is the subject whose visual acuity is being assessed. Consumer computing device 102 may have a touchscreen display, allowing an examiner 101 to tap the screen to send input to the device. The visual acuity examination may be presented to a device screen 103 through a graphical user interface 107 that displays various elements of the acuity examination to the user, enables examiner 101 to perform actions, and takes input from examiner 101. Consumer computing device 102 may contain a computer processor 106, which includes one or more CPUs (computer processing units). Consumer computing device 102 may also include one or more memories 105 (e.g., RAM, ROM, etc.). Memories 105 may be used to store the instructions that execute the visual acuity examination app, the input entered by an examiner 101, and results from the visual acuity examination. The visual acuity examination may be presented to a device screen 103 through a GUI that may display various elements of the acuity examination, may enable an examiner 101 to perform actions, and may take input from examiner 101. The interfaces that may be used for the presentation of this visual acuity examination are labeled as interface(s) 107. The speaker(s) that may be used for voice assistance are labeled as speaker(s) 108. A distance 104 measures the exemplary ideal distance between user 100 and computerized consumer device 102 to conduct an exemplary visual acuity examination. In one embodiment, distance 104 may approximately equal to 10 feet for distance visual acuity examination. During a visual acuity examination, consumer computing device 102 may be operated by examiner 101, who is a human assistant who does not need to be a professional in conducting vision screening. One exemplary aspect of the present invention is that examiner 101 may be holding consumer computing device 102, as illustrated by FIG. 1.

FIG. 2 illustrates another exemplary setting for performing a computerized visual acuity examination, which is similar to FIG. 1. However, FIG. 2 depicts another exemplary aspect of the invention, in which consumer computing device 102 is placed on a surface 201 so that examiner 101 does not need to hold the device. Although FIG. 2 illustrates surface 201 as a table, any surface that can support the device may suffice.

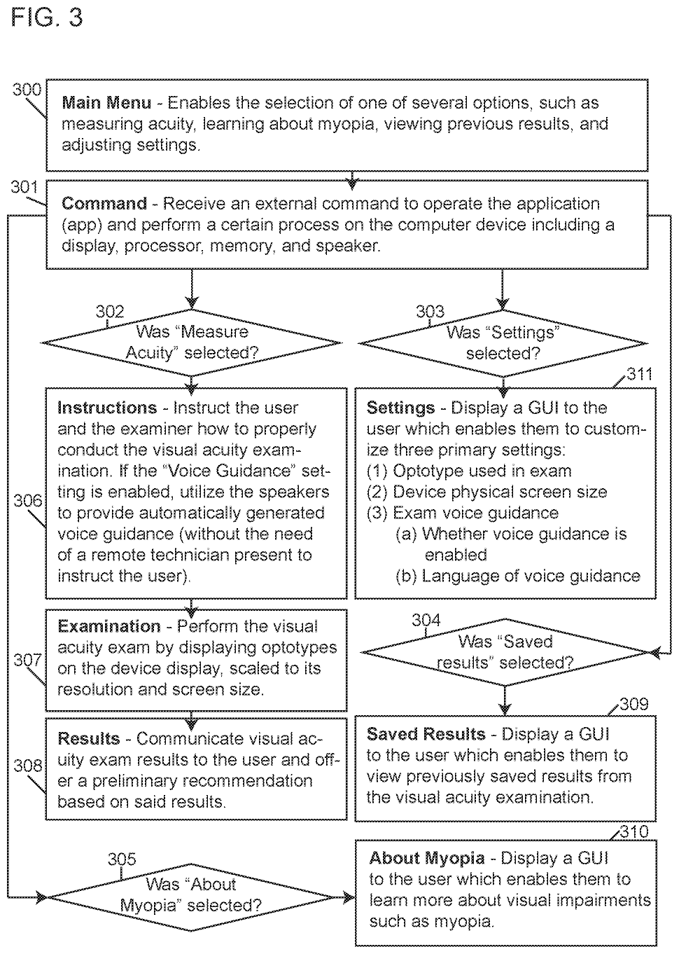

Exemplary approaches and GUIs that may be utilized by a visual acuity exam application (app) will now be described through FIGS. 3-13.

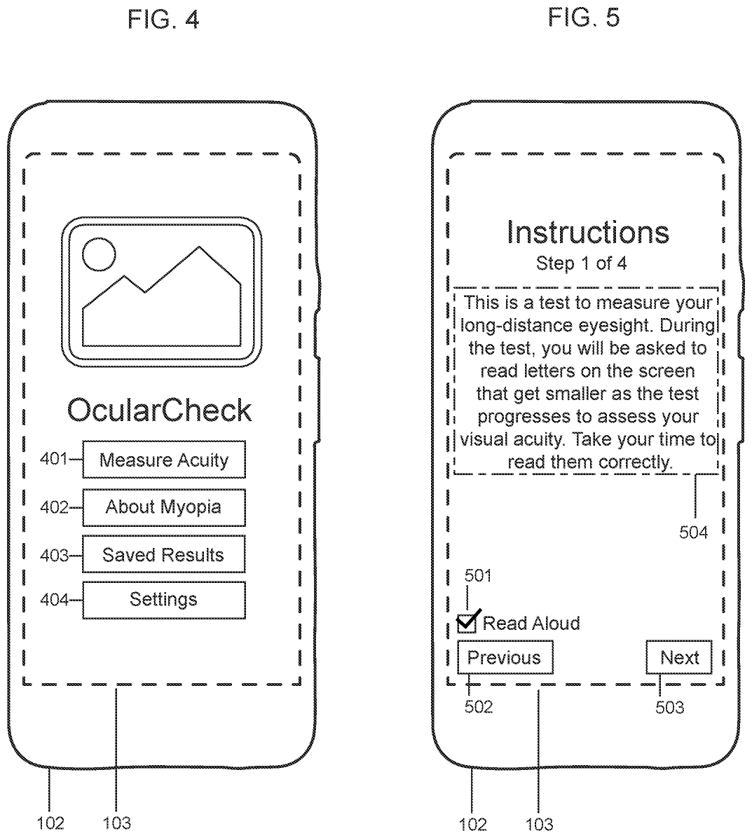

FIG. 3 illustrates a flow diagram for an exemplary approach for an application that performs computerized visual acuity examinations in a way that solves the problems discussed in the Background section. In this exemplary approach, the visual acuity examination is conducted through an app, the instructions of which are stored on consumer computing device 102. As shown in a step 300 and a step 301, consumer computing device 102 may receive a command from user 100 or examiner 101 to perform one of the following procedures, comprising of the following list: a step 302 (“measure acuity”), a step 305 (“about myopia”), a step 304 (“saved results”), or a step 303 (“settings”). An exemplary GUI in which computerized consumer device 102 receives this command input is illustrated in FIG. 4, in which buttons 401-404 represent the four aforementioned commands, respectively.

If examiner 101 selects a button 401, as shown by a step 302, the app may present a GUI, as exemplarily illustrated by FIG. 5, showing a series of instructions (a step 306) to guide examiner 101 and user 100 on the examination procedure. The exemplary GUI of FIG. 5 includes three main components. A check box 501 allows examiner 101 to toggle whether or not these instructions are spoken aloud through speaker(s) 108. Check box 501 also may toggle whether voice assistance is enabled throughout the visual acuity examination process (including, but not limited to, the exemplary GUI illustrated by FIG. 5). Upon being selected, a button 502 may cause a panel 504 to display the previous instruction. Upon being selected, a button 503 may cause a panel 504 to display the next instruction. Once the app has finished displaying instructions on how to take the visual acuity exam, the app may proceed to a step 307.

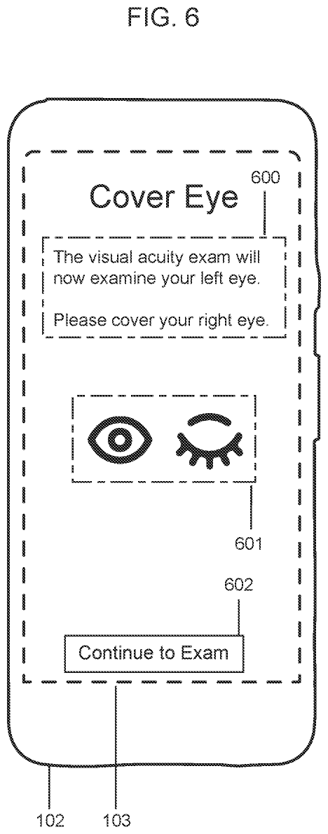

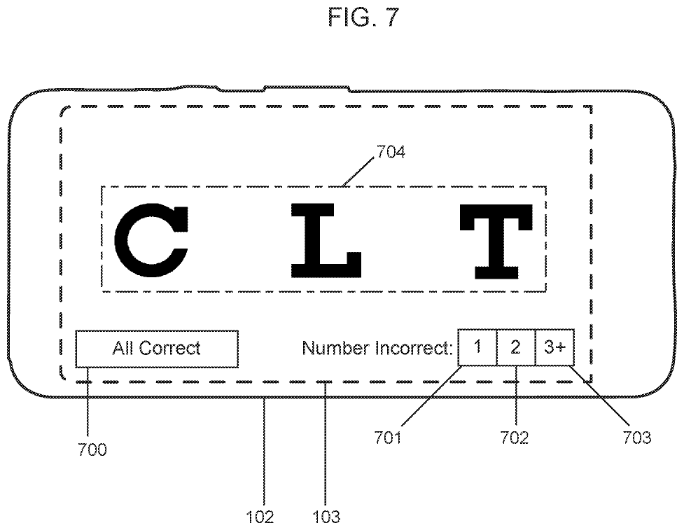

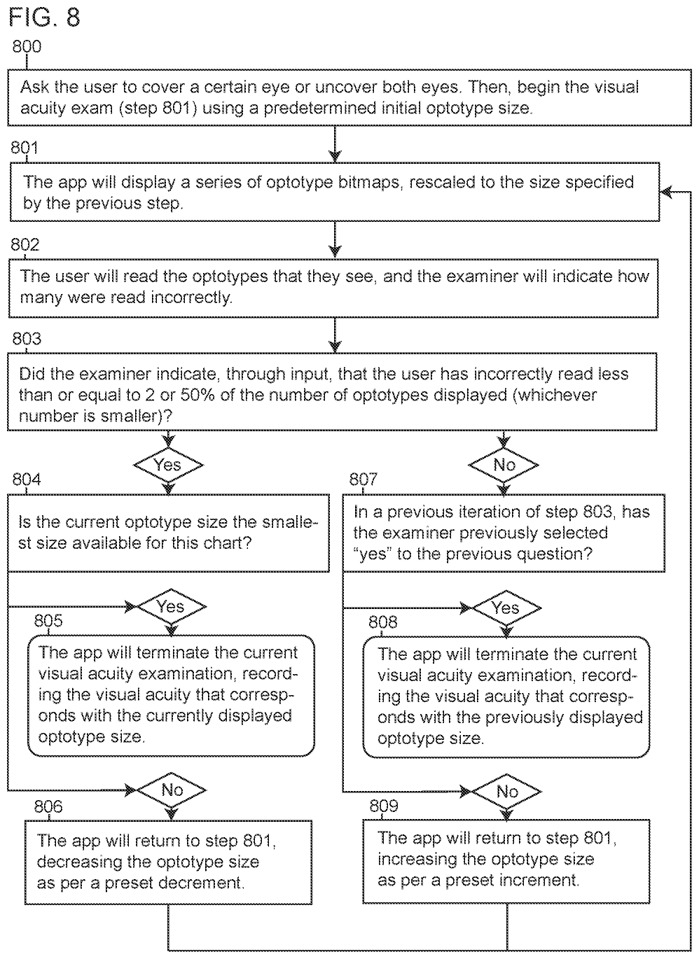

When the examination procedure (step 307) is initiated, the app may first examine user 100's right eye, then user 100's left eye, and optionally, both eyes. Optionally, this entire procedure may be repeated twice for greater accuracy. FIGS. 6 and 7 illustrate exemplary GUIs that may be displayed during step 307, and FIG. 8 illustrates a detailed flow diagram describing an exemplary approach through which these GUIs may operate to assess user 100's visual acuity.

FIG. 6 illustrates an exemplary GUI that asks user 100 to cover a certain eye or uncover both eyes before assessing their visual acuity. A text box 600 describes an exemplary instruction, in which the user is asked to cover their right eye, and a graphical image 601 provides a visualization of this. The contents of text box 600 and graphical image 601 may change, depending on which eye is currently being assessed, to reflect accurate instructions. Once a button 602 is selected, the app will continue to assess user 100's visual acuity for the eye currently under examination by presenting a GUI depicted by FIG. 7.

It should be noted that when FIG. 7 is displayed, the app may set device screen 103's display brightness to a pre-determined value (modifiable through a number-input 1007 in FIG. 10) and may disable automatic brightness adjustment in order to prevent inaccuracies caused by changing brightness levels.

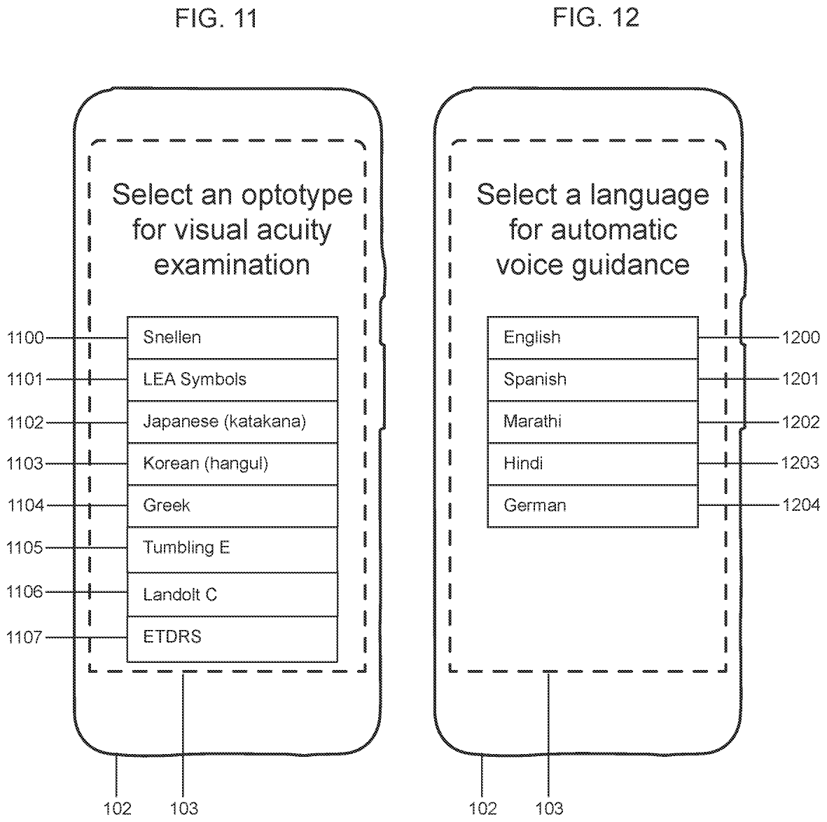

Next, FIG. 7 depicts an exemplary GUI through which the app may assess user 100's visual acuity by displaying various optotypes. A panel 704 illustrates an exemplary area in which an exemplary random sequence of optotypes may be presented as a part of the examination. The type of optotypes presented may be modified through the settings menu to select an optotype (described in the detailed description of FIG. 11). The optotypes displayed on this GUI may be scaled depending on the physical size of device screen 103 through a procedure described in the detailed description of FIG. 8. Buttons 700-703 represent various instructions that examiner 101 can select to operate the eye examination: if user 100 has identified no optotypes incorrectly, examiner 101 should select a button 700; if user 100 has identified one optotype incorrectly, examiner 101 should select a button 701; if user 100 has identified two optotypes incorrectly, examiner 101 should select a button 702; and if user 100 has identified three or more optotypes incorrectly, examiner 101 should select a button 703. After one of the buttons 700-703 has been selected, depending on which button was selected, the app may present another set of optotypes in panel 704, until the app terminates the visual acuity exam (see the detailed description of FIG. 8 for the logic the app uses to determine how to proceed). Overall, by assessing which sizes of optotypes user 100 can and cannot identify correctly, the app may determine a visual acuity measurement of the eye or set of eyes currently under examination.

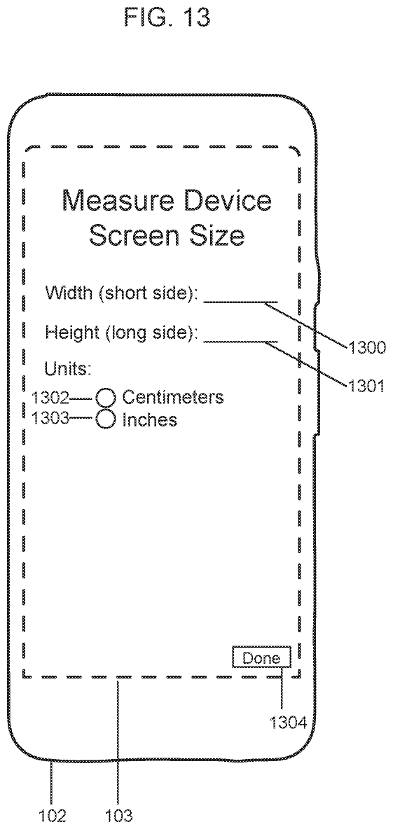

FIG. 8 illustrates a flow diagram that describes, in detail, an exemplary approach to carry out step 307 to perform a visual acuity examination for a particular eye or set of eyes. A step 800 corresponds to consumer computing device 102 displaying a GUI depicted by FIG. 6, as described earlier. Steps 801-809 correspond to consumer computing device 102 displaying a GUI depicted by FIG. 7. As a step 801 describes, optotypes 704 may be rescaled to occupy a calculated physical size. Rescaling may be performed by (1) identifying the physical size of device screen 103 either by (a) searching a database stored in the nonvolatile memory of consumer computing device 102 that lists screen dimensions using consumer computing device 102's device model number and manufacturer, or (b) by using override values provided by the user in the settings GUI (described in the detailed description of FIG. 13), (2) calculating a pixel-to-inches ratio representing how many pixels occupy 1 inch on device screen 103, and (3) multiplying this ratio by the calculated physical size to obtain the number of pixels the optotypes 704 on device screen 103. As steps 802-803 describe, user 100 may identify these optotypes, and examiner 101 may input the number of optotypes that were identified incorrectly. As steps 804-809 describe, the app may follow a set of logical IF-THEN statements to determine how to process this input from examiner 101. If the app determines that step 803 should follow the “yes” route to a step 804, then the app may determine whether it is currently displaying the smallest available optotype size. If so, as specified by step 805, the app may terminate this visual acuity examination and record a visual acuity score corresponding to the current optotype size for the current eye or set of eyes being examined. If not, the app may continue this visual acuity examination by returning to step 801 after decreasing the optotype size by a pre-determined decrement (specified by a step 806). If the app determines that step 803 should follow the “no” route to step 807, then the app may determine whether the examiner selected “yes” in any previous iterations of step 803, within the context of the visual acuity examination for the current eye or set of eyes. If so, as specified by step 808, the app may terminate this visual acuity examination and record the visual acuity score corresponding to the optotype size from the said iteration for the current eye or set of eyes being examined. If not, the app may continue this visual acuity examination by returning to step 801 after increasing the optotype size by a pre-determined increment (as specified by a step 809). The amount of increment/decrement specified by steps 806 and 809 corresponds with the difference in size of optotypes in consecutive rows of the eye chart corresponding to the type of optotype selected by button 1000.

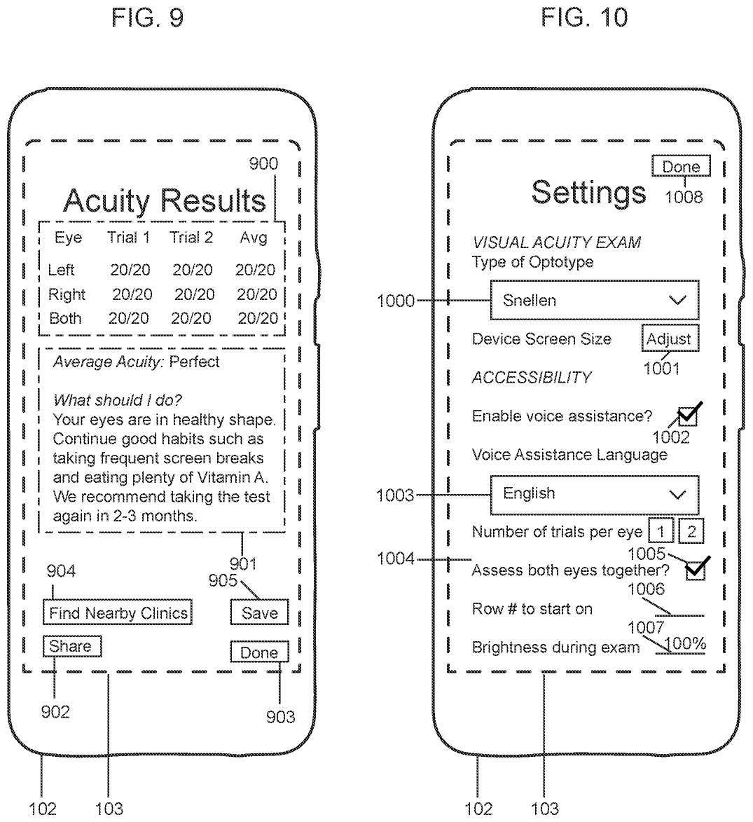

Returning to FIG. 3, once the examination procedure (step 307) is complete, the app may display a results GUI containing the results (a step 308). An exemplary results GUI is illustrated by FIG. 9. A panel 900 shows an exemplary results panel, in which three types of results may be displayed: visual acuity of left eye, visual acuity of right eye, and visual acuity of both eyes. The “visual acuity of both eyes” row is optional and may be omitted if a check box 1005 is not checked. Three columns may be shown for every eye or set of eyes examined: the acuity measurement from trial 1, the acuity measurement from trial 2, and the average of the acuity measurements in trials 1 and 2. Depending on the number of trials selected in a number-picker 1004, the columns “trial 2” and “avg” may be omitted. In one exemplary illustration, a panel 901 may be below panel 900 and may describe the implications of user 100's visual acuity results (i.e., an interpretation of their score, how to seek treatment, etc.). The contents of panels 900 and 901 may be dependent on the visual acuity exam score(s), so some elements may change between examinations depending on which eye(s) were examined. At the bottom of the GUI depicted by FIG. 9, there may be four buttons which enable examiner 101 to take various actions. A button 904 may enable examiner 101 to locate nearby professional clinics to seek diagnosis and treatment. A button 905 may save the visual acuity results (as written on panel 900) to consumer computing device 102's nonvolatile memory so that they can be accessed later. A button 902 may enable examiner 101 to send the visual acuity results (as written on panel 900) to another person(s) who may want them (i.e., their doctor, their family members, etc.). A button 903 may return to the main menu of the app (as illustrated by FIG. 4).

Returning to FIG. 3, if examiner 101 or user 100 initiates step 305 by selecting a button 402, the app may proceed to a step 310 and present a GUI which may enable them to learn more about visual impairments in an easy-to-understand, accessible manner.

Returning to FIG. 3, if examiner 101 or user 100 initiates step 304 by selecting a button 403, the app may proceed to a step 309 and present a GUI which may enable them to view visual acuity results that have been saved on the nonvolatile memory of consumer computing device 102 through the “save” feature initiated by button 705.

Returning to FIG. 3, if examiner 101 or user 100 initiates a step 303 by selecting button 404, the app may proceed to a step 311 and present a GUI which enables examiner 101 to modify various settings. An exemplary settings GUI is illustrated by FIG. 10. A button 1000 may enable examiner 101 or user 100 to change which type of optotype is used for the visual acuity examination process by presenting the exemplary GUI of FIG. 11. A button 1001 may enable examiner 101 or user 100 to provide override values for device screen 103's physical dimensions by presenting the exemplary GUI of FIG. 13. A check box 1002 may enable examiner 101 to change the default value of check box 501 when check box 501 is presented (either checked or not checked). A button 1003 may enable examiner 101 to change which language the voice assistance feature uses by presenting the exemplary GUI of FIG. 12. A number-picker 1004 may enable examiner 101 to select the number of times the visual acuity exam is performed for every eye or set of eyes. Possible inputs to number-picker 1004 may include “1” and “2”. A check box 1005 may enable examiner 101 to select whether or not both eyes are assessed together during the examination procedure. Hence, number-picker 1004 and check box 1005 may directly affect how many times the app carries out step 800 in FIG. 8. A row number-picker 1006 may enable examiner 101 to input a “row number” with which to begin each visual acuity examination. In this context, “row number” is defined in the context of the eye chart corresponding to the optotype selected by button 1000, as this eye chart contains various rows of optotypes, each of which differs in size. Ultimately, the row number entered in row number-picker 1006 is used to determine the “predetermined initial optotype size” in step 800 in FIG. 8. A number-input 1007 may allow the examiner 101 to input a percentage to control the display brightness of device screen 103 during the examination procedure. Settings modified by the interface elements 1000-1007 may be saved in the nonvolatile memory of consumer computing device 102. When a button 1008 is selected, the app may return to a main menu screen (FIG. 4). In one embodiment, button 1008 may be located in the top-right corner, but in other embodiments, button 1008 may be located in the bottom-right corner instead.

FIG. 11 illustrates an exemplary GUI for selecting the optotype used for the visual acuity examination. Buttons 1100-1107 may enable examiner 101 to select from an exemplary list comprising of various optotypes (Snellen, LEA Symbols, Japanese (katakana), Korean (Hangul), Greek, Tumbling E, Landolt C, or ETDRS). Upon clicking a button 1100-1107, the app may save the optotype selected by examiner 101 to the nonvolatile memory of consumer computing device 102 and may return to the settings GUI screen (FIG. 10).

FIG. 12 illustrates an exemplary GUI for selecting the language used for the voice assistance feature. Buttons 1200-1204 may enable examiner 101 to select from an exemplary list of various languages (comprising of English, Spanish, Marathi, Hindi, and German). Upon clicking a button 1200-1204, the app may save the language selected by examiner 101 to the nonvolatile memory of consumer computing device 102 and may return to the settings GUI screen (as illustrated by FIG. 10).

FIG. 13 illustrates an exemplary GUI for inputting the physical size of the device screen. Text input fields 1300 and 1301 may enable examiner 101 to input decimal numbers that represent the physical dimensions of device screen 103. Radio buttons 1302 and 1303 may enable examiner 101 to select between centimeters and inches for their preferred unit of input. After a button 1304 has been selected, the app may save the width, height, and units to the nonvolatile memory of consumer computing device 102 and may return to the settings GUI screen (as illustrated by FIG. 10).

The methods and systems described herein may be implemented using any suitable computer processing system with any suitable combination of hardware, software and/or firmware. The systems may include element managers, real-time data buffers, file input processors, database indices, data buffers, and data managers for managing data and processing. Consumer computing device 102 may also include multiple displays, display interfaces, input/output devices such as keyboards, microphones, mice, touch screens, and the like for permitting users to manage consumer computing device 102. This description describes in writing exemplary embodiments of the present invention. However, other variations are within the scope of the present disclosure.

The methods and systems that are described in this present disclosure may be implemented on many different types of devices that are capable of processing program code. In an implementation, the software program instructions that are used may include source code, object code, machine code, or any other stored data that can cause a processing system to execute the methods and operations that are described in this present disclosure. Any suitable computer languages may be used such as C, C++, Java, HTML, XML, etc., as well as APIs available to developers for given operating system platforms.

The systems' and methods' data (e.g., associations, mappings, data input, data output, intermediate data results, final data results, etc.) may be configured for storage using any suitable data structures, and may be stored and implemented in one or more different types of computer-implemented data stores, such as different types of storage devices and programming constructs (e.g., RAM, ROM, Flash memory, flat files, databases, programming data structures, programming variables, IF-THEN (or similar type) statement constructs, etc.). It is noted that data structures describe formats for use in organizing and storing data in databases, programs, memory, or other non-transitory computer-readable media for use by a computer program.

The computer components, software modules, functions, data stores, and data structures described herein may be connected directly or indirectly to each other to allow the flow of data needed for their operations. It is also noted that a module or processor includes but is not limited to a unit of code that performs a software operation and can be implemented for example as a subroutine unit of code, or as a software function unit of code, or as an object (as in an object-oriented paradigm), or as an applet, or in a computer script language, or as another type of computer code. The software components and/or functionality may be located on a single computer or distributed across multiple computers depending upon the situation at hand.

It should be understood that as used in the description herein and throughout the claims that follow, the meaning of “a,” “an,” and “the” includes plural reference unless the context clearly dictates otherwise. Also, as used in the description herein and throughout the claims that follow, the meaning of “in” includes “in” and “on” unless the context clearly dictates otherwise. In addition, as used in the description herein and throughout the claims that follow, the meanings of “and” and “or” include both the conjunctive and disjunctive and may be used interchangeably unless the context expressly dictates otherwise; the phrase “exclusive or” may be used to indicate situation where only the disjunctive meaning may apply.

While the present invention has been described in terms of exemplary embodiments, it may be understood by those skilled in the art that various modifications can be made thereto without departing from the scope of the invention as set forth in the claims.

What is claimed is:

1. A method for testing visual acuity of a user using a consumer computing device, the method comprising:
(a) initiating a visual acuity test to allow an examiner to assess the visual acuity of a user using a computerized consumer device, the computerized consumer device comprising a display screen, a computer processor, a speaker, and a memory, the user being a human subject and the examiner being a human assistant who does not need to be a professional in conducting vision screening;
(b) instructing the user to be located a certain distance from the consumer computing device at eye level and optionally providing automatically-generated voice assistance without the need of a remote human technician;
(c) presenting a visual acuity exam without use of a refractor lens assembly, wherein the visual acuity exam comprises displaying various optotypes on the screen of the consumer computing device for the user to identify, such that the optotypes are rescaled to occupy a calculated physical size;
(d) presenting an interface (through the consumer computing device) through which the examiner can select the number of optotypes that were identified incorrectly;
(e) repeating steps (d)-(e) between 1 and 25 times, each time modifying the optotype size based on the examiner's previous input of the number of incorrect optotypes;
(f) repeating steps (c)-(e) for each eye or set of eyes under examination, such as the right eye, the left eye, and both eyes, and optionally performing the visual acuity exam twice for each eye or set of eyes;
(g) determining visual acuity score(s), from 20/20 to 20/200, for each eye or set of eyes under examination; and
(h) presenting an interface (through the consumer computing device) comprising of the said score(s), an interpretation of said score(s), and options to locate nearby professional optometry clinics, to save the score(s) to the nonvolatile memory of the consumer computing device, and to share the score(s) with another person(s).

2. The method of claim 1, wherein the optotypes used for the visual acuity examination process can be selected through a menu comprising of Snellen, ETDRS, Japanese (katakana), Korean (Hangul), Greek, Tumbling E, LEA Symbols and Landolt C.

3. The method of claim 1, wherein the language used for voice assistance can be selected through a menu comprising of English, Spanish, German, Marathi, and Hindi.

4. The method of claim 1, wherein the optotypes used for the visual acuity examination process are rescaled in size according to the consumer computing device's screen size, such that if the user has provided override values for the device physical width and height, those values will be used, and if the user has not provided override values, then the consumer computing device will search a database of device screen sizes (which is stored in the nonvolatile memory of the consumer computing device) using the consumer computing device's model number and manufacturer as a search query.

5. The method of claim 1, wherein the consumer computing device comprises a smartphone, tablet computer, or personal computer.

6. A consumer-based system for carrying out a visual acuity test of a user, comprising: a consumer device comprising a display screen, a computer processor, a speaker, and a memory, the computer processor being configured to cause the computerized consumer device to:
(a) initiate a visual acuity test to allow an examiner to assess the visual acuity of a user, the user being a human subject and the examiner being a human assistant who does not need to be a professional in conducting vision screening;
(b) instruct the user to be located a certain distance from the consumer computing device at eye level and optionally provide automatically-generated voice assistance without the need of a remote human technician;
(c) present a visual acuity examination without use of a refractor lens assembly, wherein the visual acuity exam comprises displaying various optotypes on the screen of the consumer computing device for the user to identify, such that the optotypes are rescaled to occupy a calculated physical size;
(d) present an interface (through the consumer computing device) through which the examiner can select the number of optotypes that were identified incorrectly;
(e) repeat steps (d)-(e) between 1 and 25 times, each time modifying the optotype size based on the examiner's previous input of the number of incorrect optotypes;
(f) repeat steps (c)-(e) for each eye or set of eyes under examination, such as the right eye, the left eye, and both eyes, and optionally perform the visual acuity exam twice for each eye or set of eyes;
(g) determine visual acuity score(s), from 20/20 to 20/200, for each eye or set of eyes under examination; and
(h) present an interface (through the consumer computing device) comprising of the said score(s), an interpretation of said score(s), and options to locate nearby professional optometry clinics, to save the score(s) to the nonvolatile memory of the consumer computing device, and to share the score(s) with another person(s).

7. The system of claim 6, the computer processor being configured to cause the computerized consumer device to display optotypes used for the visual acuity examination process that can be selected by through a menu comprising of Snellen, ETDRS, Japanese (katakana), Korean (Hangul), Greek, Tumbling E, LEA Symbols and Landolt C.

8. The system of claim 6, the computer processor being configured to cause the computerized consumer device to deliver voice assistance in a language that can be selected through a menu comprising of English, Spanish, German, Marathi, and Hindi.

9. The system of claim 6, the computer processor being configured to cause the computerized consumer device to rescale in size the optotypes used for the visual acuity examination process according to the consumer computing device's screen size, such that if the user has provided override values for the device physical width and height, those values will be used and if the user has not provided override values, then the consumer computing device will search a database of device screen sizes (which is stored in the nonvolatile memory of the consumer computing device) using the consumer computing device's model number and manufacturer as a search query.

10. The system of claim 6, wherein the consumer computing device comprises a smartphone, tablet computer, or personal computer.

11. A non-transitory computer-readable medium comprising program instructions for permitting a computerized consumer device comprising a display screen, a computer processor, a speaker, and a memory to carry out a visual acuity examination, the program instructions when executed causing a computer processor of the computerized consumer device to:
(a) initiate a visual acuity test to allow an examiner to assess the visual acuity of a user, the user being a human subject and the examiner being a human assistant who does not need to be a professional in conducting vision screening;
(b) instruct the user to be located a certain distance from the consumer computing device at eye level and optionally provide automatically-generated voice assistance without the need of a remote human technician;
(c) present a visual acuity examination without use of a refractor lens assembly, wherein the visual acuity exam comprises displaying various optotypes on the screen of the consumer computing device for the user to identify, such that the optotypes are rescaled to occupy a calculated physical size;
(d) present an interface (through the consumer computing device) through which the examiner can select the number of optotypes that were identified incorrectly;
(e) repeat steps (d)-(e) between 1 and 25 times, each time modifying the optotype size based on the examiner's previous input of the number of incorrect optotypes;
(f) repeat steps (c)-(e) for each eye or set of eyes under examination, such as the right eye, the left eye, and both eyes, and optionally perform the visual acuity exam twice for each eye or set of eyes;
(g) determine visual acuity score(s), from 20/20 to 20/200, for each eye or set of eyes under examination; and
(h) present an interface (through the consumer computing device) comprising of the said score(s), an interpretation of said score(s), and options to locate nearby professional optometry clinics, to save the score(s) to the nonvolatile memory of the consumer computing device, and to share the score(s) with another person(s).

12. The non-transitory computer-readable medium of claim 11, the program instructions being configured to cause the computer processor to control the computerized computer device to display optotypes used for the visual acuity examination process that can be selected by through a menu comprising of Snellen, ETDRS, Japanese (katakana), Korean (Hangul), Greek, Tumbling E, LEA Symbols and Landolt C.

13. The non-transitory computer-readable medium of claim 11, the program instructions being configured to cause the computer processor to control the computerized computer device to deliver voice assistance in a language that can be selected through a menu comprising of English, Spanish, German, Marathi, and Hindi.

14. The non-transitory computer-readable medium of claim 11, the program instructions being configured to cause the computer processor to control the consumer computing device to rescale in size the optotypes used for the visual acuity examination process according to the consumer computing device's screen size, such that if the user has provided override values for the device physical width and height, those values will be used and if the user has not provided override values, then the consumer computing device will search a database of device screen sizes (which is stored in the nonvolatile memory of the consumer computing device) using the consumer computing device's model number and manufacturer as a search query.

15. The non-transitory computer-readable medium of claim 11, wherein the consumer computing device comprises a smartphone, tablet computer, or personal computer.
